(19) United States
(12) Patent Application Publication
(10) Pub. No.: US 2007/0059342 A1
(43) Pub. Date: Mar. 15, 2007

(54) Method and palatable pharmaceutical composition for treating herpes in animals

(76) Inventors: William C. Johns, Germantown, TN (US); William W. Miller, Starkville, MS (US)

Correspondence Address: JACOBSON HOLMAN PLLC, 400 SEVENTH STREET N.W., SUITE 600, WASHINGTON, DC, 20004, US

(21) Appl. No.: 11/224,022

(22) Filed: Sep. 13, 2005

(51) Int. Cl. A23K 1/165; A61K 31/198

(52) U.S. Cl. 424/442; 514/564

(57) ABSTRACT

An animal medication for orally treating herpes is formed of L-Lysine monohydrochloride, fishmeal and stevia sweetener.

### BACKGROUND OF THE INVENTION

1. Field of the Invention

The present invention relates to the field of Vetinary pharmaceuticals. More specifically, the invention relates to a palatable pharmaceutical for the treatment of herpes in a variety of animals including, but not limited to, horses and cats.

2. Related Art

Those of skill in the art of pharmaceuticals have proposed a variety of medications for treatment of herpes in both humans and animals. However, such proposals have not proven to be universally acceptable or superior to the prior art for a variety of reasons including not being reliably effective in curing herpes and being unpalatable to both humans and animals. Lack of palatability is a particularly acute problem with respect to animals so that force-feeding or hypodermic or intravenous injections are the only options available for using such non-palatable medications such as those included in the following discussed prior art items.

Burgard et al. Patent Application Publication No. US 2004/0062844 A1 discloses use of an acid reacting sweetener with a basic-reacting amino acid in an effort to provide a palatable medication.

Gianesllo et al. Patent Application Publication No. US 2003/0138484 A1 discloses a tablet containing lysine and a sweetener in a human medication provided in an effort to make the medication palatable to children and/or juveniles.

Hamman et al. Patent Application Publication No. US 2002/0193342 discloses masking of an unpalatable pharmaceutical component with sucralose with the composition being incorporated in beverages, foods, bars or candies.

Burgard et al. Patent Application Publication No. US 2002/0081360 A1 relates to mixing an acid-reacting sweetener with a basic-reacting amino acid for palatability purposes.

Marocchi et al. International Publication No. WO 03/035027 A1 discloses a chewable tablet said to have enhanced compliance by humans and including a vitamin, lysine or pharmaceutically acceptable salt thereof and a sweetener for masking the disgusting flavor of lysine.

Burgard et al. Japanese Patent Abstract No. JP 2002-265458 A discloses use of an L-amino acid in conjunction with an acid-reacting sweetener with a variety of sweeteners being specified for use for enhancing the palatability.

Tadahiro et al. Japanese Patent Abstract No. JP 59216551 A discloses chicken feed containing fishmeal blended with lysine.

Ford et al. Patent Application Publication No. US 2003/0153618 A1 discloses use of amino acid lysine as a topical medication for treatment of herpes simplex virus Puipe II.

Richardson et al. Patent Application Publication No. US 2002/0197313 A1 discloses a mixture of a multitude of components including L-Lysine for prevention or treatment of diseases caused by the herpes simplex virus.

Richardson et al. U.S. Pat. No. 6,632,445 B2 discloses a multi-component medication including L-Lysine for treatment of herpes simplex.

Richardson et al. U.S. Pat. No. 6,455,061 B2 discloses a medication including a multitude of components mixed with simplex virus L-Lysine for herpes treatment.

Richardson et al. U.S. Pat. No. 6,231,889 B1 discloses a multi-component herpex herpes treatment including L-Lysine mixed with a substantial number of other components.

Wilkinson International Application WO 92/15315 discloses a method of treating herpes by administering a mixture of lysine, vitamin C and hesperidin.

It is apparent that none of the above-noted prior art examples is directed to a pharmaceutical mixture of components that is both effective and palatable in the treatment of herpes in animals.

### OBJECTS OF THE INVENTION

It is accordingly a primary object of the present invention to provide an improved method for treatment of herpes in animals.

A further object of the invention is the provision of a new and improved medication for treating herpes in non-human living creatures.

Another object of the present invention is the provision of a new and improved medication for treatment of herpes in animals.

A further object of the invention is the provision of a new and improved palatable medication for treatment of herpes in animals.

Yet another object of the invention is the provision of a new and improved method of treating herpes in n on-human living creatures.

An additional object of the invention is the provision of a new and improved method of treating herpes in living creatures selected from the group comprising horses.

Other objects, features and advantages of the present invention will be apparent to those skilled in the art upon a reading of this specification including the accompanying drawings.

### SUMMARY OF THE INVENTION

The invention is in the field of animal medication and is more specifically directed to a medication and its method of use for treating herpes in animals. The medication is mixed to be palatable and consists of a mixture of lysine, fishmeal and stevia, which is orally acceptable to horses and other animals.

### DETAILED DESCRIPTION OF THE PREFERRED EMBODIMENTS

Achievement of the above objects is enabled by a preferred embodiment comprising an animal medication formed of 97.5% L-Lysine monohydrochloride, 2.0% fishmeal and 0.5% stevia. Lysine is an effective agent for treatment of herpes; however, it has an extremely bad taste, which makes it extremely difficult to be orally acceptable to animals.

However, the combination of the lysine with the fishmeal and stevia has been found to be acceptable as an oral medication by a variety of animals including horses and cats.

In describing preferred composition of the present invention, specific terminology is employed for the sake of clarity. However, the invention is not intended to be limited to the specific composition and it is to be understood that each specific composition includes all equivalents that operate in a similar manner to accomplish a similar purpose.

Modifications and variations of the above-described embodiments of the present invention are possible, as appreciated by those skilled in the art in light of the above teachings. It is therefore to be understood that, within the scope of the appended claims and their equivalents, the invention may be practiced otherwise than as specifically described.

What is claimed is:

1. A palatable pharmaceutical composition medication for treating herpes in animals comprising a mixture of L-Lysine monohydrochloride, fishmeal and a sweetener.

2. A pharmaceutical composition as recited in claim 1, wherein the sweetener is stevia.

3. A pharmaceutical composition as recited in claim 2, wherein the medication is approximately 97.5% L-Lysine monohydrochloride. 2.0% fishmeal and 0.5% stevia.

4. A method of treating herpes in animals comprising orally administering a pharmaceutical composition comprising a mixture of L-Lysine monohydrochloride fishmeal and a sweetener to an animal.

5. The method of claim 4, wherein the sweetener is stevia.

6. The method of claim 5, wherein the pharmaceutical composition is approximately 97.5% L-Lysine monohydrochloride. 2.0% fishmeal and 0.5% stevia.

7. The method of claim 4, wherein the animal is a horse.

8. The method of claim 4, wherein the animal is a cat.

9. The method of claim 7, wherein the sweetener is stevia.

10. The method of claim 9, wherein the pharmaceutical composition is approximately 97.5% L-Lysine monohydrochloride. 2.0% fishmeal and 0.5% stevia.
